(19) United States
(12) Patent Application Publication
(10) Pub. No.: US 2026/0235484 A1
(43) Pub. Date: Aug. 13, 2026

(54) ONLINE MEASUREMENT SYSTEM AND METHOD FOR GAS FLUX OF GAS-LIQUID INTERFACE

(71) Applicant: CHINA THREE GORGES CORPORATION, Wuhan, Hubei (CN)

(72) Inventors: Xinyu WAN, Wuhan (CN); Yasong CHEN, Wuhan (CN); Chong LI, Wuhan (CN); Lei CHEN, Wuhan (CN); Dianchang WANG, Wuhan (CN); Mengmeng LIU, Wuhan (CN); Lidong REN, Wuhan (CN)

(73) Assignee: CHINA THREE GORGES CORPORATION, Wuhan, Hubei (CN)

(21) Appl. No.: 19/153,530

(22) PCT Filed: Dec. 19, 2023
(86) PCT No.: PCT/CN2023/140027

§ 371 Date: Aug. 4, 2025

(30) Foreign Application Priority Data
Mar. 3, 2023 (CN) 202310202981.7

(51) Int. Cl. G01N 11/04; G01N 1/22; G01N 1/26; G01N 33/00

(52) U.S. Cl. CPC G01N 11/04; G01N 1/2205; G01N 1/26; G01N 33/0075

(57) ABSTRACT

An online measurement system and method for a gas flux of a gas-liquid interface. The online measurement system for the gas flux of the gas-liquid interface comprises: a plurality of collecting devices, a measurement device, a gas inlet pipeline, a gas outlet pipeline, a gas washing device, and a control device; each collecting device is provided with a gas inlet and a gas outlet; the measurement device is suitable for measuring the gas concentration; the gas inlet pipeline is connected to gas inlet and measurement device; gas outlet pipeline is connected to gas outlet and measurement device; gas washing device is arranged on gas outlet pipeline; and control device is in signal connection with collecting device, the measurement device, and the gas washing device. The plurality of collecting devices are configured to be connected to measurement device, and collecting devices are respectively placed at different monitoring points of gas-water interface.

### CROSS REFERENCE TO RELATED APPLICATIONS

The present application claims priority to the Chinese Patent Application with application No. 202310202981.7, entitled “Online measurement system and method for gas flux of gas-liquid interface”, field for the Chinese Patent Office on Mar. 3, 2023, the entire contents of which are hereby incorporated by reference into the present application.

### TECHNICAL FIELD

The present application relates to the field of ecological and environmental protection technology, and particularly relates to an online detection system and method for gas flux at a gas-liquid interface.

### BACKGROUND OF THE INVENTION

Global warming, a climate issue that threaten human survival and the environment, has continued to attract attention in recent years. The annual increase in greenhouse gas emissions is one of the primary factors causing global warming. Greenhouse gases mainly include CO₂, N₂O, CH₄ and other gases. The greenhouse gas emissions from water-gas interface such as rivers, reservoirs, lakes and water-related industrial facilities are crucial aspects in study the impact of human activities on the concentration of atmospheric greenhouse gases. Accurately measuring the emission rates of these greenhouse gases at the water-gas interface is the key to research in this field.

The emission intensity of greenhouse gases at the water-gas interface is generally evaluated by calculating the rate of greenhouse gas mass released per unit time from the surface of water body. For open water areas, due to the large area of the gas-liquid interface, it is impossible to capture and measure the gases released from the entire gas-liquid interface. Therefore, the flux tank method is generally used, where representative water surface monitoring points are selected to measure the greenhouse gas emission flux, and then the total greenhouse gas emissions from the water body are calculated by multiplying the flux by the total area of the gas-liquid interface.

The flux tank method utilizes a flux floating tank that floating on the water surface to keep the gas inside the tank isolated from the atmosphere. Due to mass transfer at the gas-liquid interface, the concentration of greenhouse gases within the tank gradually accumulates. By measuring the concentrations of greenhouse gases inside the tank at regular time, concentration cumulative curves of greenhouse gases over time are obtained. Afterwards, the emission fluxes of greenhouse gases at the monitoring point are calculated based on the slope of the concentration cumulative curve and the bottom area of the flux tank. Greenhouse gas concentration can be measured by both gas collection bag sampling method and online monitoring instruments method. The emission rate of greenhouse gases in water bodies exhibits significant spatial and temporal heterogeneity, due to the impact of environmental factors such as temperature, hydraulic conditions and biological distribution. Therefore, it is necessary to conduct multi-point and long-term monitoring to obtain more reliable data.

The equipment in the prior art can only determine the greenhouse gas flux at one position of the water body at a time. Multiple measurements are required when monitoring multiple points, which cannot be performed at one time, it is unable to continuously monitor gas fluxes and difficult to observe the dynamic changes in gas emissions, thereby leading to relatively large errors in measurement results.

### SUMMARY OF THE INVENTION

Therefore, the technical problem to be solved by the present application lies in overcoming the following defects in prior art: the measurement cannot be performed at one time when monitoring a plurality of points, as well as continuously monitoring of gas flux cannot be conducted, thereby leading to relatively large errors in measurement results.

To address the aforementioned problems, the present application provides an online detection system for gas flux at a gas-liquid interface, comprising:
a plurality of collection devices, each of the collection devices is provided with a gas inlet and a gas outlet;
a detection device configured to detect gas concentration;
a gas-inlet pipeline connecting the gas inlet and the detection device;
a gas-outlet pipeline connecting the gas outlet and the detection device;
a gas scrubbing device provided on the gas-outlet pipeline; and
a control device in signal connection with the collection device, the detection device and the gas scrubbing device.

Further, in this online detection system for gas flux at a gas-liquid interface, the collection device includes:
a gas collection tank having a cavity inside, wherein the bottom of the gas collection tank is in an open state and configured to be placed over a collection surface, and the gas inlet and the gas outlet are provided on the gas collection tank;
a floating body provided on the outer wall of the gas collection tank;
a gravity center adjuster provided at the bottom of the gas collection tank; and
an agitator provided inside the gas collection tank.

Further, in this online detection system for gas flux at a gas-liquid interface, the floating body is provided with a cavity inside, and a floatation bladder is provided inside the floating body.

Further, in this online detection system for gas flux at a gas-liquid interface, the collection device further includes:
a vent valve provided on the gas collection tank;
a gas flow rate detector provided on the vent valve; and
a pressure and temperature detector and a liquid level detector provided on the gas collection tank.

Further, in this online detection system for gas flux at a gas-liquid interface further includes:
a first flow dividing device connected to a plurality of the gas inlet and the gas-inlet pipeline, respectively; and
a second flow dividing device connected to the gas-outlet pipeline and a plurality of the gas outlets, respectively, and the gas scrubbing device is provided between the second flow dividing device and the collection device.

Further, in this online detection system for gas flux at a gas-liquid interface, the first flow dividing device includes a plurality of first flow dividing valve, which are configured to control the connection and disconnection between the gas collection tank and the gas-inlet pipeline;
the second flow dividing device includes a plurality of second flow dividing valve, configured to control the connection and disconnection between the gas collection tank and the gas-outlet pipeline.

Further, in this online detection system for gas flux at a gas-liquid interface, the gas scrubbing device includes:
a gas scrubbing flow dividing device provided on the gas-outlet pipeline; and
a gas scrubbing bottle connected to the gas scrubbing flow dividing device.

Further, in this online detection system for gas flux at a gas-liquid interface, further comprising:
a gas pump provided on the gas-inlet pipeline and between the first flow dividing device and the detection device;
a controller provided on the gas-inlet pipeline and between the gas pump and the detection device; and
a filter provided on the gas-inlet pipeline and between the controller and the detection device.

The present application further provides a gas online detection method using the aforementioned online detection method for gas flux at a gas-liquid interface, comprising the following steps:
step S1: system deployment: placing a plurality of the collection devices at sampling points where gases are to be collected
step S2: measurement mode selection: selecting different flux measurement modes based on actual situations;
step S3: gas scrubbing: turning on the gas scrubbing device to flush internal air of each collection device;
step S4: detection: after the flushing is completed, the collection device collects gas samples at the sampling points and delivers the collected gas to the detection device for detection; and
step S5: flux calculation: calculating gas flux based on collected data and methods under different measurement modes.

The present application has the following advantages:
1. The online detection system for gas flux at a gas-liquid interface provided by the present application includes a plurality of collection devices, a detection device, a gas-inlet pipeline, a gas-outlet pipeline, a gas scrubbing device and a control device. Each of the collection devices is provided with a gas inlet and a gas outlet. The detection device is configured to detect gas concentration. The gas-inlet pipeline is connected to the gas inlet and the detection device, and the gas-outlet pipeline is connected to the gas outlet and the detection device. The gas scrubbing device provided on the gas-outlet pipeline. The control device is in signal connection with the collection device, the detection device and the gas scrubbing device.
By connecting a plurality of collection devices with the detection device and placing each of the collection devices at different monitoring points of water-gas interface, respectively, a plurality of points may be monitored simultaneously. This setup eliminates the need for frequently disassemble and reassemble the collection devices, thereby reducing operator-induced errors and improves overall measurement accuracy.
2. The online detection system for gas flux at a gas-liquid interface provided by the present application, wherein the collection device includes a gas collection tank, a floating body, a gravity center adjuster and an agitator. The gas collection tank is provided with a cavity inside, the bottom of the gas collection tank is in an open state and configured to be placed over a collection surface. The gas inlet and the gas outlet are provided on the gas collection tank, the floating body is provided on the outer wall of the gas collection tank, the gravity center adjuster is provided at the bottom of the gas collection tank and the agitator is provided inside the gas collection tank.
By providing a gravity center adjuster at the bottom of the gas collection tank, the gas collection tank may be placed at the water-gas interface more stably, thereby preventing gas spillover from the bottom opening of the gas collection tank and improving the accuracy of collection and detection results. This configuration may also be applied to areas with relatively large water surface disturbances, thereby enhancing the accuracy of monitoring.
3. The online detection system for gas flux at a gas-liquid interface provided by the present application, wherein the gas scrubbing device includes a gas scrubbing flow dividing device provided on the gas-outlet pipeline and a gas scrubbing bottle connected to the gas scrubbing flow dividing device.
By providing a gas scrubbing bottle, the residual gas from the previous round of flux measurement in the system may be flushed clean, allowing for the start of a new round of gas measurement, thereby achieving continuous online monitoring of gas flux.
4. The online detection system for gas flux at a gas-liquid interface provided by the present application, the sampling device includes including a gas flowmeter and a vent valve, connected to a control device.
By controlling the vent valve switch through the control device, switching between two measurement modes, cumulative slope method and concentration-flow volume method, may be achieved, thereby adapting to different gas detection environments.
5. The online detection method for gas flux at a gas-liquid interface provided by the present application includes the following steps:
step S1: system deployment: placing a plurality of collection devices at sampling points for gas collection;
step S2: measurement mode selection: selecting different flux measurement modes based on actual situations;
step S3: gas scrubbing: turning on the gas scrubbing device to flush internal air of each collection device (1);
step S4: detection: after the flushing is completed, the collection device collects gas samples at the sampling points and delivers the collected gas to the detection device (2) for detection; and
step S5: flux calculation: calculating the gas flux based on collected data and methods under different measurement modes.

By connecting a plurality of collection devices with the detection device and placing each of the collection devices at different monitoring points of the water-gas interface separately, a plurality of points may be monitored simultaneously without the need to disassemble and assemble the collection devices frequently for multiple measurements, thereby reducing errors in measurement results caused by anthropogenic factors during disassembly and assembly and improving the accuracy of detection. This configuration allows for switching between two measurement methods: cumulative slope method and concentration-flow volume method, thereby adapting to different gas detection environments. By providing a gas scrubbing bottle, the residual gas from the previous round of flux measurement in the system may be flushed clean during detection, thereby enabling a new round of gas measurement and achieving continuous online monitoring of gas flux.

### DESCRIPTION OF THE DRAWINGS

To illustrate the specific embodiments of the present application or the technical solutions in prior art more clearly, a brief introduction to the accompanying drawings required for description of the specific embodiments or prior art will be given below. It is obvious that the accompanying drawings in the following description are some embodiments of the present application. For those skilled in the art, other drawings may be obtained according to these accompanying drawings without creative labor.

FIG. 1 is a schematic diagram of an online detection system for gas flux at a gas-liquid interface provided in the embodiment of the present application;

FIG. 2 is a front view of the collection device in the online detection system for gas flux at a gas-liquid interface provided in the embodiment of the present application;

FIG. 3 is a top view of the collection device in the online detection system for gas flux at a gas-liquid interface provided in the embodiment of the present application;

FIG. 4 is a schematic diagram of the opening and closing of the first flow dividing valve and the second flow dividing valve under the concentration cumulative slope measurement mode in the online detection method for gas flux at a gas-liquid interface provided in the embodiment of the present application;

FIG. 5 is a schematic diagram of the opening and closing of the gas scrubbing flow dividing valve under the concentration cumulative slope measurement mode in the online detection method for gas flux at a gas-liquid interface provided in the embodiment of the present application;

FIG. 6 is a schematic diagram of the opening and closing of the first flow dividing valve and the second flow dividing valve under the concentration-flow volume measurement mode in the online detection method for gas flux at a gas-liquid interface provided in the embodiment of the present application.

### EXPLANATION OF REFERENCE SIGNS

1. Collection device; 11. Gas inlet; 12. Gas outlet; 13. Gas collection tank; 14. Floating body; 141. Floatation bladder; 15. Gravity center adjuster; 16. Agitator; 171. Gas flow rate detector; 172. Pressure and temperature detector; 173. Liquid level detector; 18. Vent valve; 2. Detection device; 3. Gas scrubbing device; 31. Gas scrubbing flow dividing device; 32. Gas scrubbing bottle; 4. Control device; 5. First flow dividing device; 51. First flow dividing valve; 6. Second flow dividing device; 61. Second flow dividing valve; 7. Gas pump; 8. Controller; 9. Filter.

### DETAILED DESCRIPTION OF THE SPECIFICATION

The following will provide a clear and complete description of the technical solution of the present application in conjunction with the accompanying drawings. Obviously, the described embodiments are a part of the embodiments of the present application, not all embodiments. Based on the embodiments in the present application, all other embodiments obtained by those ordinary skilled in the art without contributing creative labor pertain to the protection scope of the present application.

In the description of the present application, it should be noted that the terms “center”, “up”, “down”, “left”, “right”, “vertical”, “horizontal”, “inside”, “outside” etc., indicate the orientation or positional relationship based on the orientation or positional relationship shown in the accompanying drawings. These terms are only used for the convenience of describing the present application and simplifying the description, and do not indicate or imply that the devices or elements referred to must have a specific orientation or be constructed and operated in a specific orientation. Therefore, it cannot be understood as a limitation on the present application. In addition, the terms “first”, “second” and “third” are only used for description and cannot be understood as indicating or implying the relative importance.

In the description of the present application, it should be noted that unless otherwise explicitly specified and limited, the terms “mount”, “connect”, and “communicate” should be understood in a broad sense. For example, it may be fixed connection, detachable connection, or integral connection; it may be mechanical connection or electrical connection; it may be directly connected, indirectly connected through an intermediate medium, or communication between the interior of two elements. For those ordinary skilled in the art, the specific meanings of the aforementioned terms in the present application may be understood based on the specific situations.

In addition, the technical features involved in different embodiments of the present application described below may be combined with each provided they are not in conflict.

### Embodiment 1

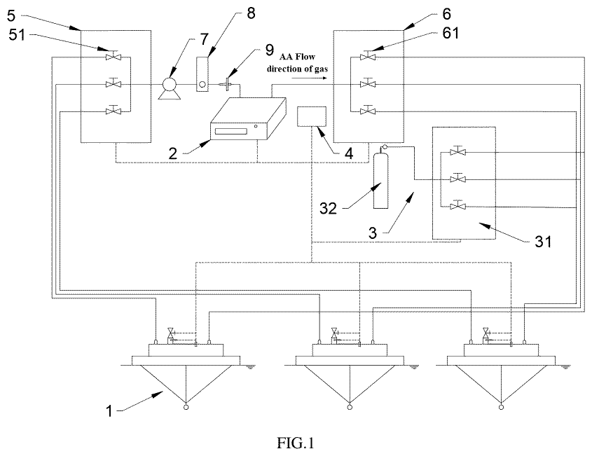

As shown in FIGS. 1 to 3, is the online detection system for gas flux at a gas-liquid interface provided by this embodiment, including: a plurality of collection devices 1, detection device 2, a gas-inlet pipeline, a gas-outlet pipeline, a gas scrubbing device 3 and a control device 4. Each of the collection devices 1 is provided with a gas inlet 11 and a gas outlet 12. The detection device 2 is configured to detect gas concentration. The gas-inlet pipeline is detachably connected to the gas inlet 11 and the detection device 2, and the gas-outlet pipeline is detachably connected to the gas outlet 12 and the detection device 2. The gas scrubbing device 3 is provided on the gas-outlet pipeline. The control device 4 is in signal connection with the collection devices 1, detection device 2 and gas scrubbing device 3.

This embodiment does not specifically limit the control device 4. To conform to practical situations, the control device 4 in this embodiment includes: a PLC controller, a displayer and a data memory. Wherein, the PLC controller is in signal connection with the collection devices 1, the detection device 2, the gas scrubbing device 3, the displayer and the data memory. The PLC controller may be set to different working modes, such as concentration cumulative slope mode and concentration-flow volume mode, and may also continuously monitor the dynamic changes in greenhouse gas emission rates over a long period of time.

By connecting a plurality of collection devices 1 to the detection device 2 and placing each of the collection devices 1 at different monitoring points on the water-gas interface separately, a plurality of points may be monitored simultaneously without the need to disassemble and assemble the collection devices frequently for multiple measurements, thereby reducing errors in measurement results caused by anthropogenic factors during disassembly and assembly and improving the accuracy of detection.

In this embodiment, by adding or reducing the gas collection device 1, the number of simultaneous monitoring points may be freely selected to meet the monitoring requirements of different environments.

Each of the collection devices 1 includes a gas collection tank 13, a floating body 14, a gravity center adjuster 15 and an agitator 16. The gas collection tank 13 is provided with a cavity inside, its bottom is in an open state, and it is configured to be placed over a collection surface. The gas inlet 11 and the gas outlet 12 are provided on the gas collection tank 13. The floating body 14 is provided on the outer wall of the gas collection tank 13. The gravity center adjuster 15 is provided at the bottom of the gas collection tank 13. The agitator 16 is provided inside the gas collection tank 13 and in signal connection with the control device 4.

By providing a gravity center adjuster 15 at the bottom of the gas collection tank 13, the gas collection tank may be placed at the water-gas interface more stably, thereby preventing gas spillover from the bottom opening of the gas collection tank 13 and improving the accuracy of collection and detection results. This configuration may also be applied to areas with relatively large water surface disturbances, thereby enhancing the accuracy of monitoring.

This embodiment does not specifically limit the gravity center adjuster 15. To conform to practical situations, the gravity center adjuster 15 in this embodiment is a relatively heavy ball, which is connected to the bottom of the floating body 14 via a connecting rope. After installation, the ball is constantly kept in a position below the center of the gas collection tank 13, thereby ensuring that the gravity center of the entire collection device 1 is located below the water surface. In areas with relatively large hydraulic disturbances, the gravity center may be automatically adjusted, thus allowing the gas collection tank 13 to closely adhere to the water surface.

The present embodiment does not specifically limit the gas collection tank 13. To conform to practical situations, the gas collection tank 13 in the present embodiment is in a cylindrical shape. In some other unshown embodiments, the gas collection tank 13 may also be set as a rectangular structure.

In the present embodiment, the pipe of the gas collection tank 13, the gas-inlet pipeline and the gas-outlet pipeline are made of PTFE material, which may increase the airtightness of the system.

In the present embodiment, the floating body 14 is in circular shape and provided with a cavity inside. The floating body 14 is fitted and mounted on the outer wall of the gas collection tank 13, and the bottom surface of the floating body 14 is at the same horizontal level as the opening on the bottom surface of the gas collection tank 13. A floatation bladder 141 filled with gas is provided inside the floating body 14. By providing the floatation bladder 141, the buoyancy of the gas collection tank 13 may be increased, thereby enabling the gas collection tank 13 to float on the collection surface of the water body more stably.

In the present embodiment, gas within the gas collection tank 13 may be stirred and mixed evenly by providing an agitator 16, thereby enhancing the accuracy of the detection results.

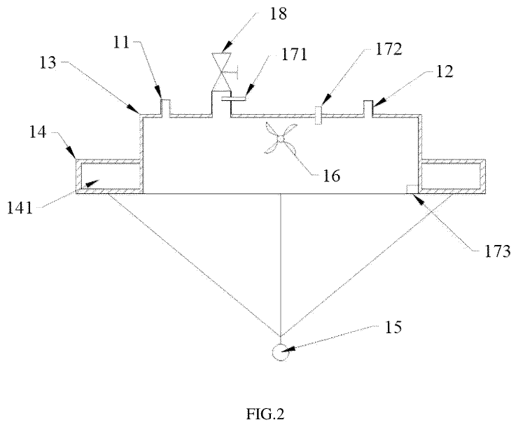

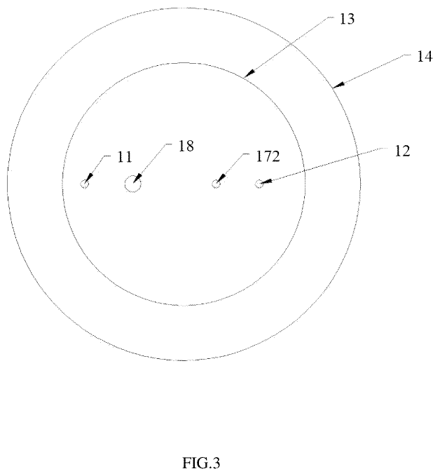

As shown in FIGS. 2 and 3, each of the collection devices 1 further includes: a vent valve 18 provided on the top of the gas collection tank 13; a gas flow rate detector 171 provided on the vent valve 18; a gas pressure and temperature detector 172 provided on the top of the gas collection tank 13; and a liquid level detector 173 provided on the inner wall of the gas collection tank 13 and located at the opening.

As shown in FIG. 1, in the present embodiment, the system further includes a first flow dividing device 5 and a second flow dividing device 6. The first flow dividing device 5 is connected to a plurality of gas inlets 11 and the gas-inlet pipeline, respectively, while the second flow dividing device 6 is connected to the gas-outlet pipeline and a plurality of gas outlets 12, respectively. The gas scrubbing device 3 is provided between the second flow dividing device 6 and the collection devices 1.

The first flow dividing device 5 includes a plurality of first flow dividing valves 51 provided in parallel, which are configured to control the connection and disconnection between the gas collection tank 13 and the gas-inlet pipeline. The inlets of the first flow dividing valves 51 are communicated with the gas inlet 11 of the sampling device through gas-guide pipes, respectively, and the outlets of the first flow dividing valves 51 are merged and connected to the detection device 2. The second flow dividing device 6 includes a plurality of second flow dividing valves 61 provided in parallel, which are configured to control the connection and disconnection between the gas collection tank 13 and the gas-outlet pipeline. The inlets of the second flow dividing valves 61 are communicated with the detection device 2, and the outlets of the second flow dividing valves 61 are communicated with the gas outlet 12 of the collection devices 1, respectively. Wherein, the first flow dividing valves 51 and the second flow dividing valves 61 are in signal connection with the control device 4, respectively, for controlling the opening and closing of the first flow dividing valves 51 and the second flow dividing valves 61.

As shown in FIG. 1, in the present embodiment, the gas scrubbing device 3 includes a gas scrubbing flow dividing device 31 provided on the gas-outlet pipeline and a gas scrubbing bottle 32 connected to the gas scrubbing flow dividing device 31.

By providing a gas scrubbing device to flush the internal gas of the collection devices 1, the initial gas concentration may be reset under the concentration cumulative slope measurement mode, thereby reducing the impact of residual gas on the detection results and improving the accuracy of the test results.

The present embodiment does not specifically limit the gas scrubbing bottle 32. To conform to practical situations, the gas scrubbing bottle 32 used in the present embodiment is a compressed air bottle. A pressure reducing valve is mounted at the outlet of the gas scrubbing bottle 32. In some other embodiments not shown, the compressed air bottle may be replaced by an air compressor or an air pump 7.

The present embodiment does not specifically limit the gas scrubbing flow dividing device 31. To conform to practical situations, the gas scrubbing flow dividing device 31 in the present embodiment includes a plurality of gas scrubbing flow dividing valves provided in parallel. The inlet of each gas scrubbing flow dividing valve is connected to the pressure reducing valve, and the outlet of each gas scrubbing flow dividing valve is communicated with the gas-outlet pipeline, respectively.

In the present embodiment, the first flow dividing valve 51, the second flow dividing valve and the gas scrubbing flow dividing valve all adopt solenoid valves, which are in signal connection with the PLC controller for controlling the opening and closing of the solenoid valves.

As shown in FIG. 1, in the present embodiment, the system further includes a gas pump 7, a controller 8 and a filter 9. The gas pump 7 is provided on the gas-inlet pipeline and located between the first flow dividing device 5 and the detection device 2. The outlets of each first flow dividing valves 51, which are provided in parallel, are merged and communicated with the gas pump 7. The controller 8 is provided on the gas-inlet pipeline and located between the gas pump 7 and the detection device 2, and is configured to control the flow rate of gas. The filter 9 is provided on the gas-inlet pipeline and located between the controller 8 and the detection device 2.

The present embodiment does not specifically limit the controller 8. To conform to practical situations, a rotameter is employed as the controller 8 in the present embodiment.

The present embodiment does not specifically limit the filter 9. To conform to practical situations, a filter membrane of 0.45 microns is provided on the filter 9 in the present embodiment, thereby enabling both gas filtration and flow rate control.

In summary, when using the online detection system for gas flux at a gas-liquid interface provided in the present embodiment, a plurality of collection devices 1 are first placed at sampling points on the water surface where gas to be collected. Then, the gravity center adjuster 15 is suspended at the bottom of the collection devices 1 to sink into water, so that the bottom opening of the gas collection tank 13 is completely placed over the water surface. After the operator selects the sampling mode (concentration cumulative slope mode or concentration-flow volume mode), the gas samples in each gas collection device 1, under the control of the first flow dividing device 5, enter the detection device 2 through the gas pump 7 and the controller 8 in sequence to measure the greenhouse gas concentration. Then, the gas samples return to the original gas collection device 1 through the second flow dividing device 6. The PLC controller automatically reads the data from each detector on the collection devices 1 and stores it in the data memory. By selecting different measurement mode, the greenhouse gas emission flux under the corresponding measurement mode is calculated separately. Then, based on the surface area of the sampled water body, the greenhouse gas emission rate of the entire water body may be calculated.

### Embodiment 2

The online detection method for gas flux at a gas-liquid interface provided in the present embodiment uses the online detection method for gas flux at a gas-liquid interface in embodiment 1, comprising the following steps:
step S1: system deployment: a plurality of the collection devices (1) is placed at sampling points where gases are to be collected
step S2: measurement mode selection: different flux measurement modes are selected based on actual situations;
step S3: gas scrubbing: the gas scrubbing device (3) is turned on to flush the internal air of each collection device (1);
step S4: detection: after the flushing is completed, gas samples are collected at the sampling points by the collection devices (1), and the collected gas is delivered to the detection device (2) for detection; and
step S5: flux calculation: the gas flux is calculated based on collected data and methods under different measurement modes.

By connecting a plurality of collection devices 1 with the detection device 2 and placing each of the collection devices 1 at different monitoring points of the water-gas interface, respectively, a plurality of points may be monitored simultaneously without the need to disassemble and assemble the collection devices 1 frequently for multiple measurements, thereby reducing errors in measurement results caused by anthropogenic factors during disassembly and assembly and improving the accuracy of detection. The present configuration allows for switching between two measurement methods: cumulative slope method and concentration-flow volume method, thereby adapting to different gas detection environments. By providing a gas scrubbing bottle, the residual gas from the previous round of flux measurement in the system may be flushed clean during detection, thereby enabling a new round of gas measurement and achieving continuous online monitoring of gas flux.

The present embodiment does not specifically limit the sampling modes. To conform to practical situations, the sampling modes in the present embodiment include concentration cumulative slope mode or concentration-flow volume mode.

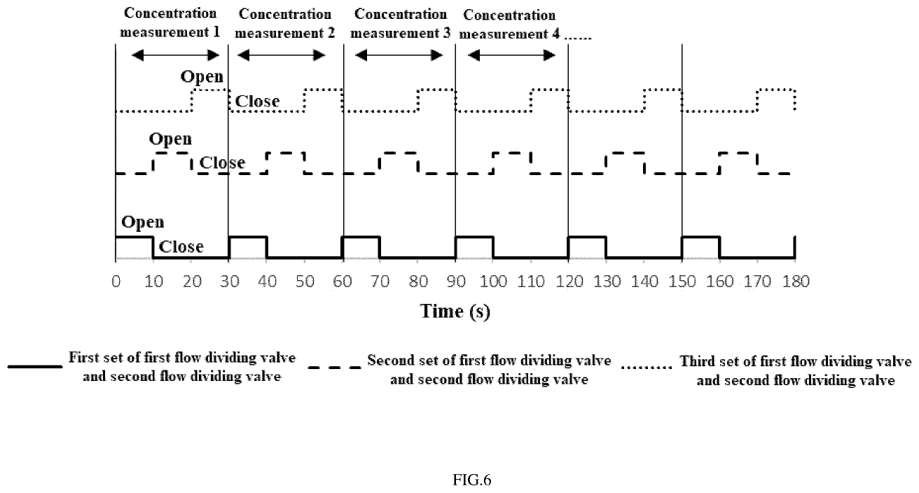

As shown in FIG. 6, in the present embodiment, the measurement method of the concentration cumulative slope mode is as follows: first, each gas collection devices 1 is placed at sampling points on a water surface with a low gas dissipation rate (such as lakes, rivers, reservoirs, anaerobic zones of sewage treatment plants, etc.). Next, the gravity center adjuster 15 is suspended under the gas collection and sampling system to sink into the water. Afterwards, the operator selects the concentration cumulative slope measurement mode. The gas scrubbing device first flushes the internal gas of each collection device 1, after the flushing is completed, the gas samples in each collection device 1, under the control of the first flow dividing device 5, enter the detection device 2 through the gas pump 7, the controller 8 and the filter 9 in sequence to measure the greenhouse gas concentration. Then, the gas samples return to the collection devices 1 through the second flow dividing device 6. The control device 4 automatically reads the data from each detector on the collection devices 1 and stores it in the data memory. After all samples in each collection device 1 are collected once in turn, the next set of gas concentration measurements begins with the first gas collection device 1. Such cycle continues until the end of the entire sampling period. The system calculates the gas emission flux of each collection device 1 during the sampling period based on the slope of concentration change over time, temperature, pressure and other data within each collection device 1. Subsequently, the internal gas of each collection device 1 is flushed by the gas scrubbing device, and then the next period of monitoring is carried out according to the aforementioned steps.

The present embodiment does not specifically limit the detection points. To conform to practical situations, a continuous online monitoring method is employed in the present embodiment, which automatically monitors the 24-hour emission data of greenhouse gases at three points in the anaerobic zone of the sewage treatment plant, with emission data being detected once every hour.

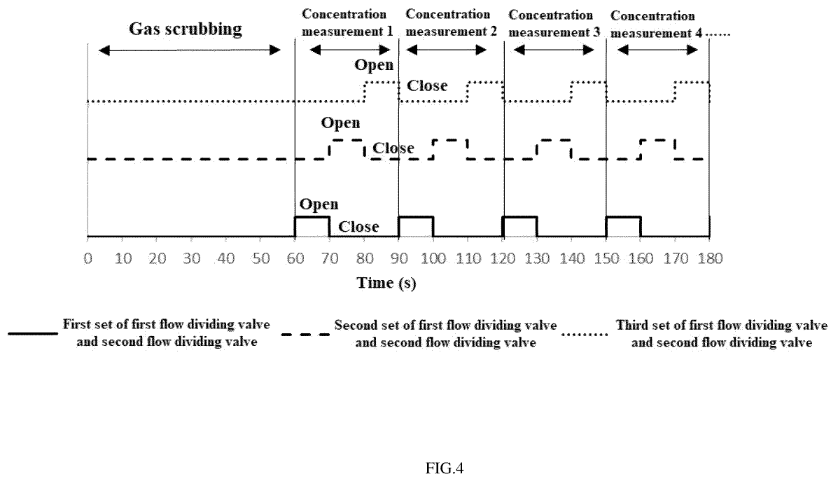

The specific steps are as follows:
step S1: deployment of collection devices 1: first, each gas collection device 1 is placed at each sampling point, allowing each gas collection device 1 to float on the water surface; then, each gravity center adjuster 15 is suspended under the collection devices 1 to sink into the water, the agitator 16 is turned on, and each detector begins to monitor and record data such as temperature, pressure and liquid level at each point.
step S2: gas scrubbing: all vent valves 18 on each of the collection devices 1 are opened, all first flow dividing valves 51 and second flow dividing valves 61 are closed, and the gas scrubbing flow dividing valve is fully opened; the internal air of each gas collection device 1 is flushed by the gas scrubbing device at a flow volume of 1 L/s; after flushing for 60 seconds, the gas concentration detection step is initiated;
step S3: measurement of concentration cumulative slope: the gas pump 7 is turned on, and the system begins to sequentially measure the concentration within each collection device 1; the specific control is as shown in FIG. 4, during measurement, the gas scrubbing flow dividing valve connected to the measured collection device 1 is closed (and remains closed thereafter), the first flow dividing valve 51 and the second flow dividing valve 61 are opened, the gas is directed into the detection device 2 for greenhouse gas concentration measurement and then returned to the collection device 1; after 10 seconds of measurement, the first flow dividing valve 51 and the second flow dividing valve 61 corresponding to the currently measured collection device 1 are closed, and then the system switches to measure the next gas collection device 1; after the collection of all collection devices 1 is completed in turn, the aforementioned steps are repeated to measure the next gas concentration in each collection devices 1 (while keeping the gas scrubbing flow dividing valve closed) until the accumulated concentration data from each collection device 1 meets the precision requirements for calculating the concentration-time cumulative slope; the concentration-time cumulative slope is calculated by linear fitting of the measured concentrations at different time points.

In the present embodiment, the number of measured concentrations at each point is set to exceed 10. To conform to practical situations, the number of measured concentrations at each point in the present embodiment is set to 20. It takes 7 minutes to complete a single detection of the concentration cumulative slope.
step S4: long-term continuous multiple detections: after completing step S3 each time, the gas scrubbing step of step S2 is repeated, and then step S3 is repeated for measurements of the next set of concentration-cumulative slope; in the present embodiment, preferably, different continuous detection frequencies may be established according to the sampling plan; after each step S3, a waiting period may be allowed before starting the next concentration-time cumulative slope measurement, during which the vent valve 18 on each collection device 1 is opened; the greenhouse gas dissipation flux at each point may be calculated according to the following formula:

$${F}_{i , j} = \frac{{k}_{i , j} \times {V}_{i} \times M \times {P}_{i , j}}{{A}_{i} \times 8.314 \times ( 273.15 + {T}_{i , j} )}$$

Wherein, F_{i,j} represents the flux at the i-th measurement point at the j-th moment, k represents the concentration-time cumulative curve slope (ppm/s) of greenhouse gas in the i-th sampling tank at the j-th moment, Vᵢ represents the volume (m³) of the i-th gas collection tank, Aᵢ represents the contact area (m²) between the i-th sampling tank and the water body, T_{i,j} represents the temperature (° C.) at the i-th sampling point at the j-th moment, P_{i,j} represents the pressure (KPa) at the i-th sampling point at the j-th moment, and M represents the relative molecular mass (g/mol) of the measured greenhouse gas.

Preferably, in the present embodiment, a wait time of 53 minutes is implemented after each step S3.
step S5: end of measurement: after completing the monitoring of each sampling point, the data collection and recording are terminated and the data are exported; the collection devices 1 is removed from the water surface and cleaned, all first flow dividing valves 51 and second flow dividing valves 61 are opened, the gas pump 7 is maintained in operation for 20 seconds to flush residual gas in the detection device 2 and pipelines using air; subsequently, the system components are organized and stored properly; in the present embodiment, preferably, the dynamic variation of overall greenhouse gas emission rate from the measured water body may be calculated based on flux data from each measurement point:

$${E}_{j} = {CA}_{T} {\sum }_{i = 1}^{n} {F}_{i , j}$$

Wherein, A_{T} represents the total surface area (m²) of the monitored pool, F_{i,j} represents the flux (gm⁻²s⁻¹) at the i-th sampling point at the j-th moment, C is the time unit conversion constant (3600 s/h), and E represents the greenhouse gas emission rate (gh⁻¹) of the monitored water body at the j-th moment.

In the present embodiment, the measurement method of the concentration-flow volume mode is as follows: first, each collection device 1 is placed at sampling points on a water surface with a relative high gas dissipation rate (such as the aerobic zone of a sewage treatment plant). Next, the gravity center adjuster 15 is suspended under the gas collection sampling system to sink into the water. Afterwards, the operator selects the concentration-flow volume measurement mode. The gas samples in each collection device 1, under the control of the first flow dividing device 5, enter the detection device 2 through the gas pump 7 and the controller 8 in sequence to measure the greenhouse gas concentration. Then, the gas samples return to the collection devices 1 through the second flow dividing device. The PLC controller automatically reads the data from each detector on the collection devices 1 and stores it in the data memory. After all samples in each collection device 1 are collected once in turn, the next set of gas concentration measurements begins with the first collection device 1. Such cycle continues until the end of the entire sampling period. The PLC controller stores the data from the detector of each collection device 1 in the data memory. The system calculates the real-time gas emission flux of each gas collection device 1 during the sampling period based on the data such as concentration and gas flow rate in each collection device 1.

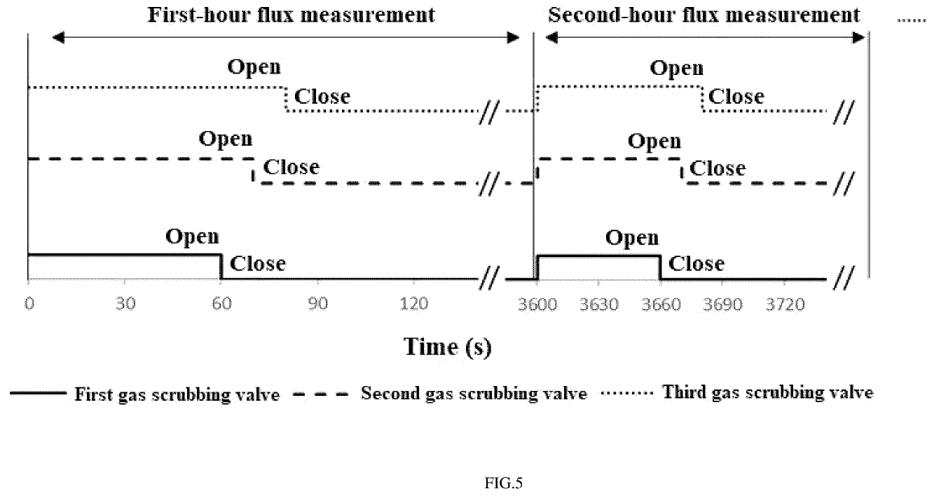

The specific steps are as follows:
Step S1: deployment of collection device 1: each collection device 1 is placed at each sampling point, allowing each collection device 1 to float on the water surface; each gravity center adjuster 15 is suspended under the collection devices 1 to sink into the water, the agitator 16 and the vent valve 18 are turned on while all gas scrubbing flow dividing valves are closed; and each detector begins to monitor and record data such as temperature, pressure, gas flow rate of the vent valve 18 and liquid level at each point.
step S2: concentration measurement: air samples are collected to measure the greenhouse gas concentration C₀ in the atmospheric background; the gas pump 7 is turned on, and the system begins to sequentially measure the concentration within each collection device 1; as shown in FIG. 5, during measurement, the first flow dividing valve 51 and the second flow dividing valve 61 connected to the measured collection devices 1 are opened, the gas is directed into the detection device 2 for greenhouse gas concentration measurement and then returned to the collection devices 1; after 10 seconds of measurement, the first flow dividing valve 51 and the second flow dividing valve 61 corresponding to the current measured collection device 1 are closed, and then the system switches to measure the next collection device 1; after the collection of all collection devices 1 is completed in turn, the aforementioned steps are repeated to measure the next gas concentration in each collection devices 1 (while keeping the gas scrubbing flow dividing valves closed) until the entire sampling is completed; the greenhouse gas dissipation flux at each point may be calculated according to the following formula:

$${F}_{i , j} = \frac{{Q}_{i , j} \times ( {C}_{i , j} - {C}_{0} ) \times {V}_{i} \times M \times {P}_{i , j}}{{A}_{i} \times 8.314 \times ( 273.15 + {T}_{i , j} )}$$

Wherein, F_{i,j} represents the greenhouse gas emission flux (gm⁻²s⁻¹) at the i-th measurement point at the j-th moment, Q_{i,j} represents the vent port flow volume (m³s⁻¹) at the i-th measurement point at the j-th moment, C_{i,j} represents the greenhouse gas concentration (ppm) at the i-th measurement point at the j-th moment, C₀ represents the background value (ppm) of greenhouse gas concentration in the atmosphere, Vᵢ represents the volume (m³) of the i-th gas collection tank 13, Aᵢ represents the contact area (m²) between the i-th sampling tank and the water body, T_{i,j} represents the temperature (C) at the i-th measurement point at the j-th moment, P_{i,j} represents the atmospheric pressure (KPa) at the i-th measurement point at the j-th moment, and M is the relative molecular mass (g/mol) of the measured greenhouse gas.
step S3: end of measurement: after completing the monitoring of each sampling point, the data collection and recording are terminated and the data are exported; the collection devices 1 is removed from the water surface and cleaned, all first flow dividing device valves 51 and second flow dividing device valves 61 are opened, the gas pump 7 is maintained in operation for 20 seconds to flush residual gas in the detection device 2 and pipelines using air; subsequently, the system components are organized and stored properly; in the present embodiment, preferably, the dynamic variation of overall greenhouse gas emission rate from the measured water body may be calculated based on flux data from each measurement point:

$${E}_{j} = {CA}_{T} {\sum }_{i = 1}^{n} {F}_{i , j}$$

Wherein, A_{T} represents the total surface area (m²) of the monitored pool, F_{i,j} represents the flux (gm⁻²s⁻¹) at the i-th sampling point at the j-th moment, C is the time unit conversion constant (3600 s/h), and E represents the greenhouse gas emission rate (gh⁻¹) of the monitored water body at the j-th moment.

In summary, the gas online detection method in the present embodiment may achieve simultaneous measurement of multiple water surface points, thereby increasing the amount and scope of data collection while minimizing labor and material costs, significantly improving data accuracy. The present method achieves long-term and continuous online monitoring of gas flux as well as dynamic monitoring of greenhouse gas emissions, thus reducing the cost of frequent manual collection and simultaneously improving measurement accuracy.

In the present embodiment, different measurement modes may be switched according to the gas dissipation rate of the measured water body to improve the measurement accuracy in corresponding situations. When the gas dissipation rate from the measured water body (such as in lakes, reservoirs, anaerobic zones of sewage treatment plants, etc.) is relatively low, a concentration cumulative rate measurement mode is employed to measure the gas dissipation flux. Conversely, when the gas dissipation rate of the measured water body (such as aeration zones of sewage treatment plants) is relatively high, it may be switched to a measurement method that measures the gas concentration within the tank and the dissipated gas flow volume, thereby expanding the detection applicability range.

Obviously, the embodiments described above are only embodiments provided for clear illustration, and are not intended to limit the embodiments. For those ordinary skilled in the relevant field, other forms of changes or variations may be made based on the above description. It is neither necessary nor possible to exhaust all embodiments here. The obvious changes or variations arising from the present are still within the protection scope of the present application.

1. An online detection system for gas flux at a gas-liquid interface, comprising:
a plurality of collection devices, wherein each of the collection devices is provided with a gas inlet and a gas outlet;
a detection device configured to detect gas concentration;
a gas-inlet pipeline connecting the gas inlet and the detection device;
a gas-outlet pipeline connecting the gas outlet and the detection device;
a gas scrubbing device provided on the gas-outlet pipeline; and
a control device in signal connection with the collection device, the detection device and the gas scrubbing device.

2. The online detection system for gas flux at a gas-liquid interface according to claim 1, wherein the collection device comprises:
a gas collection tank having a cavity inside, wherein bottom of the gas collection tank is in an open state and configured to be placed over collection surface, and the gas inlet and the gas outlet are provided on the gas collection tank;
a floating body provided on the outer wall of the gas collection tank;
a gravity center adjuster provided at bottom of the gas collection tank; and
an agitator provided inside the gas collection tank.

3. The online detection system for gas flux at a gas-liquid interface according to claim 2, wherein the floating body is provided with a cavity inside, and a floatation bladder is provided inside the floating body.

4. The online detection system for gas flux at a gas-liquid interface according to claim 3, wherein the collection device further comprises:
a vent valve provided on the gas collection tank;
a gas flow rate detector provided on the vent valve; and
a pressure and temperature detector and a liquid level detector provided on the gas collection tank.

5. The online detection system for gas flux at a gas-liquid interface according to claim 2, further comprising:
a first flow dividing device connected to a plurality of the gas inlets and the gas-inlet pipeline, respectively; and
a second flow dividing device connected to the gas-outlet pipeline and a plurality of the gas outlets, respectively, and the gas scrubbing device is provided between the second flow dividing device and the collection device.

6. The online detection system for gas flux at a gas-liquid interface according to claim 5, wherein:
the first flow dividing device comprises a plurality of first flow dividing valves, configured to control the connection and disconnection between the gas collection tank and the gas-inlet pipeline; and
the second flow dividing device comprises a plurality of second flow dividing valves, configured to control the connection and disconnection between the gas collection tank and the gas-outlet pipeline.

7. The online detection system for gas flux at a gas-liquid interface according to claim 5, wherein the gas scrubbing device comprises:
a gas scrubbing flow dividing device provided on the gas-outlet pipeline; and
a gas scrubbing bottle connected to the gas scrubbing flow dividing device.

8. The online detection system for gas flux at a gas-liquid interface according to claim 6, further comprising:
a gas pump provided on the gas-inlet pipeline and between the first flow dividing device and the detection device;
a controller provided on the gas-inlet pipeline and between the gas pump and the detection device; and
a filter provided on the gas-inlet pipeline and between the controller and the detection device.

9. An online detection method for gas flux at a gas-liquid interface using the online detection system for gas flux at a gas-liquid interface according to claim 1, comprising the following steps:
step S1: system deployment: placing a plurality of the collection devices at sampling points where gases are to be collected;
step S2: measurement mode selection: selecting different flux measurement modes based on actual situations;
step S3: gas scrubbing: turning on the gas scrubbing device to flush internal air of each collection device;
step S4: detection: after the flushing is completed, the collection device collects gas samples at the sampling points and delivers the collected gas to the detection device for detection; and
step S5: flux calculation: calculating the gas flux based on collected data and methods under different measurement modes.

10. The online detection system for gas flux at a gas-liquid interface according to claim 3, further comprising:
a first flow dividing device connected to a plurality of the gas inlets and the gas-inlet pipeline, respectively; and
a second flow dividing device connected to the gas-outlet pipeline and a plurality of the gas outlets, respectively, and the gas scrubbing device is provided between the second flow dividing device and the collection device.

11. The online detection system for gas flux at a gas-liquid interface according to claim 4, further comprising:
a first flow dividing device connected to a plurality of the gas inlets and the gas-inlet pipeline, respectively; and
a second flow dividing device connected to the gas-outlet pipeline and a plurality of the gas outlets, respectively, and the gas scrubbing device is provided between the second flow dividing device and the collection device.

12. The online detection system for gas flux at a gas-liquid interface according to claim 7, further comprising:
a gas pump provided on the gas-inlet pipeline and between the first flow dividing device and the detection device;
a controller provided on the gas-inlet pipeline and between the gas pump and the detection device; and
a filter provided on the gas-inlet pipeline and between the controller and the detection device.
